(19) United States
(12) Patent Application Publication
(10) Pub. No.: US 2008/0069876 A1
(43) Pub. Date: Mar. 20, 2008

(54) Method for preparing solid dosage forms that form a paste, cream or gel when diluted for final use

(76) Inventor: Ryan G. Moore, Lilburn, GA (US)

Correspondence Address: WITHERS & KEYS, LLC, P. O. BOX 2049, MCDONOUGH, GA, 30253, US

(21) Appl. No.: 11/901,120

(22) Filed: Sep. 14, 2007

Related U.S. Application Data
(60) Provisional application No. 60/825,698, filed on Sep. 14, 2006.

(51) Int. Cl. A61K 9/20; B65D 85/00

(52) U.S. Cl. 424/464; 206/528; 424/49; 424/70.1

(57) ABSTRACT

Solid compositions capable of forming a paste, cream or gel suitable for use as a personal hygiene product when diluted with a liquid are disclosed. Methods of making and using solid compositions capable of forming a paste, cream or gel suitable for use as a personal hygiene product when diluted with a liquid are also disclosed.

### CROSS-REFERENCE TO RELATED APPLICATIONS

This patent application claims the benefit of priority to U.S. provisional patent application Ser. No. 60/825,698 entitled “METHOD FOR PREPARING SOLID DOSAGE FORMS THAT FORM A PASTE, CREAM OR GEL WHEN DILUTED FOR FINAL USE”, filed on Sep. 14, 2006, the subject matter of which is hereby incorporated by reference in its entirety.

### FIELD OF THE INVENTION

The present invention relates generally to solid compositions that are capable of forming a paste, cream or gel suitable for use as a personal hygiene product when diluted with a liquid such as water. The present invention further relates to methods of making and using solid compositions that are capable of forming a paste, cream or gel suitable for use as a personal hygiene product when diluted with a liquid.

### BACKGROUND OF THE INVENTION

Many personal hygiene products such as toothpaste, oral care products, shaving products, shampoo, hair conditioner, hair gel, creams and lotions are available to the consumer in the form of pre-packaged gels, pastes or liquids. There are a number of disadvantages associated with such liquid-containing products. For example, recent air travel restrictions limit the total volume of liquid in a given container to no more than 88.7 milliliters (3.0 U.S. ounces). Such limitations require the air traveler to either minimize the amount of pre-packaged gels, pastes or liquids or travel without these types of products.

There is a need in the art for pre-formulated solid particulate personal hygiene products that can be converted into a paste, cream or gel suitable for use as a personal hygiene product when diluted with a liquid such as water.

### SUMMARY OF THE INVENTION

The present invention is directed to pre-formulated solid particulate personal hygiene products that can be converted into a paste, cream or gel suitable for use as a personal hygiene product when diluted with a liquid such as water. The pre-formulated solid particulate personal hygiene products comprise a combination of ingredients, which result in a particulate personal hygiene product in the form of one or more tablets, one or more compressed granules, or a plurality of loose powder particles. When diluted with a liquid, such as water, the particulate personal hygiene product converts into a personal hygiene product in the form of a gel, paste, lotion or cream having desired properties such as a desired product viscosity and consistency, one or more active ingredients, and optional flavors and/or fragrance.

Accordingly, the present invention is directed to particulate personal hygiene products. In one exemplary embodiment of the present invention, the particulate personal hygiene product is in the form of one or more tablets, one or more compressed granules, or a plurality of loose powder particles, and comprises at least one thickening agent in an amount greater than 10 wt %; at least one active ingredient in an amount ranging from greater than 0 wt % to about 50 wt %, the at least one active ingredient being selected from the group consisting of a tooth cleaner, a tooth whitener, a breath freshener, an antimicrobial agent, a foaming agent, a surfactant, a hair conditioning agent, an anti-itch agent, a moisturizing agent, a sun-blocking agent, and combinations thereof; optionally, at least one flavoring agent in an amount up to about 2.0 wt %; optionally, at least one fragrance-producing agent in an amount up to about 2.0 wt %; wherein all weight percents are based on a total weight of the particulate personal hygiene product, and wherein the particulate personal hygiene product (i) forms a gel, paste or cream when blended with a predetermined amount of liquid, and (ii) comprises less than about 5.0 wt % water.

The present invention is further directed to personal hygiene products in the form of a gel, paste or cream formed from particulate personal hygiene products of the present invention. Such personal hygiene products formed from particulate personal hygiene products of the present invention include, but are not limited to, toothpaste, an oral care product, a shaving product, shampoo, hair conditioner, hair gel, skin lotion or skin cream.

The present invention is even further directed to packaged solid personal hygiene products. In one exemplary embodiment of the present invention, the packaged solid personal hygiene product comprises particulate personal hygiene product capable of forming toothpaste, an oral care product, a shaving product, shampoo, hair conditioner, hair gel, skin lotion or skin cream when blended with a predetermined amount of liquid, the particulate personal hygiene product being in the form of one or more tablets, one or more compressed granules, or a plurality of loose powder particles; and packaging material that encloses and protects the particulate personal hygiene product. The packaging material may be a simple envelope or pouch formed from a film material or a more complex container comprising, for example, a molded bottle portion (e.g., a pill bottle) with a molded cap portion.

The present invention is even further directed to methods of making personal hygiene products. In one exemplary embodiment of the present invention, the method of making a personal hygiene product comprises forming particulate personal hygiene product in the form of one or more tablets, one or more compressed granules, or a plurality of loose powder particles, wherein the particulate personal hygiene product is capable of forming toothpaste, an oral care product, a shaving product, shampoo, hair conditioner, hair gel, skin lotion or skin cream when blended with a predetermined amount of liquid. The exemplary method of making a personal hygiene product may further comprise a number of additional steps including, but not limited to, packaging the particulate personal hygiene product in packaging material that encloses and protects the particulate personal hygiene product, and combining a predetermined amount of liquid with the particulate personal hygiene product to form a personal hygiene product comprising a gel, paste or cream.

The present invention is even further directed to methods of providing personal hygiene products. In one exemplary embodiment of the present invention, the method of providing a personal hygiene product comprises providing a packaged solid personal hygiene product comprising (i) particulate personal hygiene product in the form of one or more tablets, one or more compressed granules, or a plurality of loose powder particles within (ii) a packaging material that encloses and protects the particulate personal hygiene product, the particulate personal hygiene product being convertible into a personal hygiene product when combined with a predetermined amount of liquid such as water.

The present invention is even further directed to methods of using personal hygiene products. In one exemplary embodiment of the present invention, the method of using a personal hygiene product comprises opening a packaged solid personal hygiene product comprising (i) particulate personal hygiene product in the form of one or more tablets, one or more compressed granules, or a plurality of loose powder particles within (ii) a packaging material that encloses and protects the particulate personal hygiene product, the particulate personal hygiene product being convertible into a personal hygiene product when combined with a predetermined amount of liquid such as water; and combining the predetermined amount of liquid with the particulate personal hygiene product to form a personal hygiene product comprising a gel, paste or cream. The exemplary method of using a personal hygiene product may further comprise a number of additional steps including, but not limited to, removing the particulate personal hygiene product from the packaging material; removing the gel, paste or cream from the packaging material; storing an unused portion of the gel, paste or cream in the packaging material or some other storage container; and applying the gel, paste or cream onto at least one of teeth, hair and skin.

These and other features and advantages of the present invention will become apparent after a review of the following detailed description of the disclosed embodiments and the appended claims.

### DETAILED DESCRIPTION OF THE INVENTION

The present invention is directed to solid particulate compositions capable of forming a gel, paste or cream suitable for use as a personal hygiene product when combined with a liquid such as water. The present invention is further directed personal hygiene product in the form of a gel, paste or cream, wherein the personal hygiene product is formed from a solid particulate composition in combination with a liquid, such as water. The present invention is even further directed to methods of making and using solid particulate compositions capable of forming a gel, paste or cream suitable for use as a personal hygiene product when combined with a liquid such as water, as well as the resulting personal hygiene product.

### I. Particulate Personal Hygiene Products

The solid particulate personal hygiene products of the present invention are in the form of tablets, compressed granules, or a plurality of loose powder particles. Further, the solid particulate personal hygiene products of the present invention may comprise a number of individual components. A description of individual components and combinations of individual components is provided below.

A. Particulate Personal Hygiene Product Components

The particulate personal hygiene products of the present invention may comprise one or more of the following components.

1. Thickening or Gelling Agents

The particulate personal hygiene products of the present invention typically comprise at least one thickening or gelling agent. Suitable thickening or gelling agent for use in the present invention include, but are not limited to, polyvinylpyrollidone, alkali metal starches, alkali metal starch glycolates, carboxycellulose compounds, cellulosic gums, xanthan gums, locust bean gums, and all other naturally-occurring gums and starches. As used herein, the above-mentioned thickening or gelling agents include both crosslinked and uncrosslinked versions of the thickening or gelling agents. For example, any of the above-mentioned starches, gums, cellulosic-based compounds, or polymers may be crosslinked via bonding forces within a given compound or polymer or via the addition of one or more crosslinking agents.

The particulate personal hygiene products of the present invention typically comprise one or more thickening or gelling agents in an amount greater than 10 weight percent (wt %) based on a total weight of the particulate personal hygiene product. In some exemplary embodiments, the particulate personal hygiene products comprise one or more thickening or gelling agents in an amount ranging from about 10 wt % to about 99.9 wt %, more typically, from about 15 wt % to about 99.5 wt %, and even more typically, from about 40 wt % to about 99 wt %, based on a total weight of the particulate personal hygiene product.

A number of commercially available thickening or gelling agents may be used in the present invention. Suitable commercially available thickening or gelling agents include, but are not limited to, crosslinked potato starch commercially available from Campina Corp. (Netherlands) and sold under the trade designation PRIMOJEL®; and hydroxypropyl methylcellulose commercially available from Dow Chemical (Midland, Mich.) and sold under the trade designation METHOCEL™ such as METHOCEL™ 40-202.

In one desired embodiment, the thickening or gelling agent comprises a sodium starch glycolate prepared from potato starch such as PRIMOJEL® crosslinked sodium starch glycolate.

2. Active Ingredients

The particulate personal hygiene products of the present invention also comprise at least one active ingredient typically used to formulate personal hygiene products such as toothpastes, oral care products, shaving products, shampoos, hair conditioners, hair gels, skin lotions, or skin creams. Suitable active ingredients for use in the present invention include, but are not limited to, a tooth cleaner, a tooth whitener, a breath freshener, an antimicrobial agent, a foaming agent, a surfactant, a hair conditioning agent, an anti-itch agent, a moisturizing agent, an emollient, an anti-aging agent, an anti-wrinkle agent, a sun-blocking agent, and combinations thereof.

Each active ingredient may be present in an amount ranging from greater than 0 to about 50 wt % based on a total weight of the particulate personal hygiene product. In some exemplary embodiments, each active ingredient is present in a given particulate personal hygiene product in an amount ranging from about 0.001 wt % to about 90 wt %, more typically, from about 0.05 wt % to about 10 wt %, and even more typically, from about 0.1 wt % to about 5.0 wt %, based on a total weight of the particulate personal hygiene product.

A number of commercially available active ingredients may be used in the present invention. Suitable commercially available active ingredients include, but are not limited to, emollients such as emollients commercially available from Croda Inc. (Edison, N.J.) under the trade designation CRODALAN AWS™, and emollients commercially available from Stepan (Northfield, Ill.) under the trade designation 745 GC (i.e., a glyceryl cocoate), as well as emollient-related products such as octyl palminate commercially available from Stepan (Northfield, Ill.); lubricants such as glycerine, and lubricants commercially available from Croda Inc. (Edison, N.J.) under the trade designation CRODALAN AWS™; foam enhancers such as foam enhancers commercially available from Stepan (Northfield, Ill.) under the trade designation NINOL®; moisturizers such as Jojoba oil commercially available from Arista (Wilton, Conn.); surfactants such as sodium lauryl sulfate commercially available from Stepan (Northfield, Ill.); and teeth protecting agents such as sodium fluoride commercially available from Univar USA (Bellevue, Wash.).

3. Flavoring Agents

The particulate personal hygiene products of the present invention may further comprise one or more flavoring agents and sweeteners. Suitable flavoring agents include, but are not limited to, mint flavors (e.g., spearmint or peppermint flavor), orange flavor, cherry flavor, lime flavor, bubble gum flavor, etc. Suitable sweetening agents include, but are not limited to, glucose, sorbitol, sucralose, neo-tame, potassium acesulfame, etc. In one exemplary embodiment of the present invention, the particulate personal hygiene products of the present invention comprise either a spearmint or peppermint flavoring agent and/or sucralose as the sweetener.

When present, each of the one or more flavoring agents and sweeteners may be present in a given particulate personal hygiene product in an amount of up to about 2.0 wt % based on a total weight of the particulate personal hygiene product. Typically, each of the one or more flavoring agents are independently present in a given particulate personal hygiene product in an amount ranging from about 0.0010 to about 1.75 wt %, more desirably from about 0.020 to about 1.5 wt %, and even more desirably from about 0.05 to about 1.25 wt % based on a total weight of the particulate personal hygiene product.

A number of commercially available flavoring agents may be used in the present invention. Suitable commercially available flavoring agents include, but are not limited to, peppermint flavoring syrup and spearmint flavoring syrup commercially available from Arylessence (Marietta, Ga.). A number of commercially available sweetening agents may be used in the present invention. Suitable commercially available flavoring agents include, but are not limited to, xylitol, sucralose, neotame and ACE-K commercially available, for example, from Univar USA (Bellevue, Wash.).

4. Fragrance-Producing Agents

The particulate personal hygiene products of the present invention may further comprise one or more fragrance-producing agents. Suitable fragrance-producing agents include, but are not limited to, lemon, orange, mint, pine, green-apple, eucalyptus, jasmine and various spa-like fragrances. In one exemplary embodiment of the present invention, the particulate personal hygiene products of the present invention comprise either a green-apple or eucalyptus fragrance agent.

When present, each of the one or more fragrance agents may be present in a given particulate personal hygiene product in an amount of up to about 2.0 wt % based on a total weight of the particulate personal hygiene product. Typically, each of the one or more fragrance agents are independently present in a given particulate personal hygiene product in an amount ranging from about 0.0010 to about 1.75 wt %, more desirably from about 0.020 to about 1.5 wt %, and even more desirably from about 0.25 to about 1.25 wt % based on a total weight of the particulate personal hygiene product.

A number of commercially available flavoring agents may be used in the present invention. Suitable commercially available flavoring agents include, but are not limited to, Green-Apple Fragrance commercially available from Arylessence (Marietta, Ga.).

5. Colorants

The particulate personal hygiene products of the present invention may further comprise one or more colorants. Suitable colorants include, but are not limited to, FD&C Blue #1, FD&C Green #3, FD&C Yellow #5 and FD&C Red #40. In one exemplary embodiment of the present invention, the particulate personal hygiene products of the present invention comprise either a FD&C Blue #1 or FD&C Yellow #5.

When present, each of the one or more colorants may be present in a given particulate personal hygiene product in an amount of up to about 1.0 wt % based on a total weight of the particulate personal hygiene product. Typically, each of the one or more colorants are independently present in a given particulate personal hygiene product in an amount ranging from about 0.0010 to about 0.0050 wt %, more desirably from about 0.0020 to about 0.0035 wt %, and even more desirably from about 0.0025 to about 0.0028 wt % based on a total weight of the particulate personal hygiene product.

A number of commercially available colorants may be used in the present invention. Suitable commercially available colorants include, but are not limited to, FD&C Blue #1, FD#C Green #3, FD&C Yellow #5 and FD&C Red #40 commercially available from Prime Ingredients (Saddle Brook, N.J.).

6. Production Processing Aids

The particulate personal hygiene products of the present invention may further comprise one or more production processing aids. Suitable production processing aids include, but are not limited to, polyethylene glycols, alkali metal carbonates, alkali metal bicarbonates, malto-dextrins, etc. In one exemplary embodiment of the present invention, the particulate personal hygiene products of the present invention comprise either polyethylene glycol or sodium bicarbonate.

When present, each of the one or more processing aids may be present in a given particulate personal hygiene product in an amount of up to about 30 wt % based on a total weight of the particulate personal hygiene product. Typically, each of the one or more processing aids are independently present in a given particulate personal hygiene product in an amount ranging from about 0.1 to about 15 wt %, more desirably from about 0.5 to about 10 wt %, and even more desirably from about 1.0 to about 8.0 wt % based on a total weight of the particulate personal hygiene product.

A number of commercially available processing aids may be used in the present invention. Suitable commercially available processing aids include, but are not limited to, CARBOWAX™ 8000 commercially available from Dow Chemical (Midland, Mich.), PEG 3350 commercially available from Union Carbide (Houston, Tex.), LUTROL® 6000 commercially available from BASF (Florham Park, N.J.), and MALTRIN® M510 commercially available from Grain Processing Corporation (Muscatine, Iowa).

7. Other Additives

The particulate personal hygiene products of the present invention may further comprise one or more additives in order to provide one or more properties to the particulate personal hygiene products including, but not limited to, pH, antimicrobial properties, or any combination thereof. Suitable additives for use in the present invention include, but are not limited to, pH control agents, antimicrobial agents, etc.

Suitable pH control agents for use in the present invention include, but are not limited to, citric acid, phosphoric acid, sodium citrate, sodium benzoate, benzoic acid, sodium citrate, silver citrate, dihydrogen silver citrate, or a combination thereof. In one exemplary embodiment, the pH control agent comprises citric acid (i.e., 2-hydroxy-1,2,3-propanetricarboxylic acid), phosphoric acid, sodium benzoate, or any combination thereof.

When present, each of the one or more additives may be present in a given particulate personal hygiene product in an amount of up to about 30 wt % based on a total weight of the particulate personal hygiene product. Typically, each of the one or more additives are independently present in a given particulate personal hygiene product in an amount ranging from about 0.1 to about 15 wt %, more desirably from about 0.5 to about 10 wt %, and even more desirably from about 1.0 to about 8.0 wt % based on a total weight of the particulate personal hygiene product.

A number of commercially available additives may be used in the present invention. Suitable commercially available additives include, but are not limited to, antimicrobial Axhenol DHSC commercially available from Pure Bioscience (El Cajon, Calif.).

B. Particulate Personal Hygiene Product Structures

The particulate personal hygiene products of the present invention may be in the form of tablets, compressed granules, or a plurality of loose powder particles such as the exemplary tablets, compressed granules, and powders described below.

1. Tablets

As used herein, the term “tablet” is used to describe a solid personal hygiene product having an overall dimension greater than any dimension of any particle used to form the tablet. The tablets may have any desired size and shape. Typically, each tablet has a length, width and height dimension of at least about 5.0 millimeters (mm). In some embodiments, each tablet has length, width and height dimensions each independently ranging from about 2.0 mm to about 50 mm, more typically, from about 3.0 mm to about 30 mm, and even more typically, from about 5.0 mm to about 20 mm.

Each tablet may have any overall shape and any cross-sectional shape as measured along a height of the tablet. Suitable cross-sectional shapes include, but are not limited to, a circular shape, a rectangular shape, a triangular shape, a square shape, etc. In one exemplary embodiment, each tablet has an overall cylindrical shape and a circular cross-sectional area. In this exemplary embodiment, each tablet has a diameter ranging from about 10 mm to about 20 mm, and a height ranging from about 3.0 to about 12 mm.

Tablet may be formed using conventional tablet-forming methods.

2. Compressed Granules

As used herein, the term “compressed granules” is used to describe a solid personal hygiene product having an overall dimension greater than any dimension of any particle used to form a given compressed granule, but less than the above-described dimensions of tablets. The compressed granules may have any desired size and shape. Typically, each compressed granule passes through a U.S. Standard No. 10 sieve and remains on a U.S. Standard No. 20 sieve.

Compressed granules may be formed using conventional granule-forming methods.

3. Plurality of Loose Powder Particles

As used throughout the present specification, the term “plurality of loose powder particles” refers to a powder in which individual particles are free to flow relative to one another when agitated by an external force. The term “plurality of loose powder particles” does not refer to, for example, a coating containing particulate material, a film containing particulate material, a gel, paste or cream containing particulate material, compressed granules, or tablets.

Typically, each particle within the plurality of loose powder particles will pass through a U.S. Standard No. 20 sieve.

### II. Methods of Making Particulate Personal Hygiene Product Compositions and Gels, Pastes and Creams Therefrom

The present invention is even further directed to methods of making personal hygiene products. In one exemplary embodiment of the present invention, the method of making a personal hygiene product comprises forming particulate personal hygiene product in the form of one or more tablets, one or more compressed granules, or a plurality of loose powder particles, wherein the particulate personal hygiene product is capable of forming toothpaste, an oral care product, a shaving product, shampoo, hair conditioner, hair gel, skin lotion or skin cream when blended with a predetermined amount of liquid. The exemplary method of making a personal hygiene product may further comprise combining a predetermined amount of liquid with the particulate personal hygiene product to form a personal hygiene product comprising a gel, paste or cream.

The particulate personal hygiene products of the present invention may be formed using conventional process steps. For example, solid components used to the particulate personal hygiene products may be blended with one another using standard blending techniques. If a given solid component needs to be formed into particles or if the particle size of a given solid component needs to be reduced, the solid component may be processed to reduce the particle size to a desired particle size range. When a liquid component is utilized, the liquid component may be coated onto the surface of one or more solid components. Typically, any liquid components are combined with solid components other than the gelling or thickening agent of the particulate personal hygiene product. Such a technique of combining liquid and solid components is described in U.S. Pat. No. 6,451,746 assigned to ChemLink Laboratories, Inc., the subject of which is hereby incorporated by reference in its entirety.

After blending all of the components of the particulate personal hygiene product, the final mixture of powders or particulates may be used as is or subsequently compressed into tablets or granules using conventional tablet or granule forming techniques.

Methods of making personal hygiene products of the present invention may include a number of additional method steps. Possible additional method steps may include, but are not limited to, packaging the particulate personal hygiene product in packaging material that encloses and protects the particulate personal hygiene product (e.g., from exposure to moisture); providing an amount of particulate personal hygiene product in a single unit of packaging material, wherein the amount corresponds to multiple units of personal hygiene product when combined with a predetermined amount of liquid; providing on the packaging material, for example, via printed information, the predetermined amount of liquid and a description of the type of liquid needed (e.g., water) to form a single unit of personal hygiene product (e.g., shaving cream) or to form multiple units of personal hygiene product (e.g., toothpaste, skin lotion or skin cream); forming a kit of separately packaged particulate personal hygiene products comprising two or more different particulate personal hygiene products (e.g., a toothpaste, a shaving cream, a skin lotion, etc.); providing a storage container operatively adapted for blending particulate personal hygiene product with the predetermined amount of liquid and/or storing resulting personal hygiene product for future use (i.e., for storing multiple units of toothpaste for use over a period of time such as a week); and providing on the packaging material and/or the storage container one or more markings (e.g., a line) to indicate the proper level of liquid to add to the particulate personal hygiene product in order to form a single unit of personal hygiene product (e.g., shaving cream) or to form multiple units of personal hygiene product (e.g., toothpaste, skin lotion or skin cream).

In some exemplary embodiments, the methods of making packaged solid personal hygiene product comprise packaging particulate personal hygiene product in a single package and in an amount so as to provide multiple units of resulting personal hygiene product when combined with the predetermined amount of liquid. In some embodiments, a single package of particulate personal hygiene product may form from about 1 to about 100 units, more typically, from about 6 to about 25 units, of personal hygiene product (e.g., toothpaste, shaving cream, skin lotion or skin cream) when combined with liquid.

In one exemplary embodiment, the method of making a particulate personal hygiene product comprises making a particulate personal hygiene product in the form of one or more tablets, one or more compressed granules, or a plurality of loose powder particles, wherein the particulate personal hygiene product comprises (i) at least one thickening agent in an amount greater than 10 wt %; (ii) at least one active ingredient in an amount ranging from greater than 0 to about 50 wt %, the at least one active ingredient being selected from the group consisting of a tooth cleaner, a tooth whitener, a breath freshener, an antimicrobial agent, a foaming agent, a surfactant, a hair conditioning agent, an anti-itch agent, a moisturizing agent, a sun-blocking agent, and combinations thereof; (iii) optionally, at least one flavoring agent in an amount up to about 2.0 wt %; (iv) optionally, at least one fragrance-producing agent in an amount up to about 2.0 wt %; and (v) less than about 5.0 wt % water (or less than 4.0 wt % water, or less than 3.0 wt % water, or less than 2.0 wt % water, or less than 1.0 wt % water, or less than 0.5 wt % water); wherein all weight percents are based on a total weight of the particulate personal hygiene product, and wherein the particulate personal hygiene product forms a gel, paste or cream when blended with a predetermined amount of liquid. In this exemplary method, the liquid is desirably water.

### III. Methods of Using Particulate Personal Hygiene Product Compositions and Gels, Pastes and Creams Therefrom

The present invention is further directed to methods of using the above-described particulate personal hygiene products and personal hygiene products formed therefrom. In one exemplary embodiment of the present invention, the method of using a personal hygiene product comprises opening a packaged solid personal hygiene product comprising (i) particulate personal hygiene product in the form of one or more tablets, one or more compressed granules, or a plurality of loose powder particles within (ii) a packaging material that encloses and protects the particulate personal hygiene product, the particulate personal hygiene product being convertible into a personal hygiene product when combined with a predetermined amount of liquid such as water; and combining the predetermined amount of liquid with the particulate personal hygiene product to form a personal hygiene product comprising a gel, paste or cream.

The exemplary methods of using a personal hygiene product may further comprise a number of additional steps including, but not limited to, removing the particulate personal hygiene product from the packaging material; removing the gel, paste or cream from the packaging material; storing an unused portion of the gel, paste or cream in the packaging material or some other storage container for future use; and applying the gel, paste or cream onto at least one of teeth, hair and skin.

As discussed above, in some embodiments, the method of using a personal hygiene product may comprise using a single unit of personal hygiene product. In these embodiments, a user may remove one or more tablets or one or more compressed granules from the packaging material or may measure an amount of loose powder particles (e.g., a teaspoon, an amount dictated by a pre-packaged measuring device, for example, a provided scoop, or an amount indicated on a provided storage container) and combine the desired amount of particulate personal hygiene product with a predetermined amount of liquid, such as water, to form a single unit of personal hygiene product in the form of gel, paste or cream.

In other embodiments, the method of using a personal hygiene product may comprise using multiple units of personal hygiene product. In these embodiments, a user (or multiple users) may remove one or more tablets or one or more compressed granules from the packaging material or may measure an amount of loose powder particles (e.g., one or more teaspoons, an amount dictated by a pre-packaged measuring device, for example, a provided scoop, or an amount indicated on a provided storage container) and combine the desired amount of particulate personal hygiene product with a predetermined amount of liquid, such as water, to form multiple units of personal hygiene product in the form of gel, paste or cream to be used by one or more users over a period of time (e.g., a 24 hour period, a week, a month, etc.).

The liquid added to the particulate personal hygiene product may comprise water or other hydrophilic liquids such as alcohols, glycerine, propylene glycol and polypropylene glycol. In some exemplary embodiments, the liquid comprises water. Typically, any soft or hard water may be used in the present invention, although soft water is more desirable. As used herein, the term “soft water” refers to water containing less than about 60 ppm of water hardness expressed as calcium carbonate content. As used herein, the term “hard water” refers to water containing more than about 60 ppm of water hardness expressed as calcium carbonate content, while “very hard water” refers to water containing more than about 180 ppm of water hardness expressed as calcium carbonate content. Typically, water available from any municipal water-treatment facility may be used in the present invention to form a gel, paste or cream from the particulate personal hygiene product compositions.

The amount of liquid used to form a gel, paste or cream from the particulate personal hygiene product may vary depending on a number of factors including, but not limited to, the type of product (e.g., toothpaste, shaving cream, etc.), and the number of units desired. Typically, the amount of liquid used to form a given unit of gel, paste or cream from the particulate personal hygiene product ranges from about 5.0 milliliters (ml) to about 50 ml, more typically, form about 5.0 ml to about 20 ml.

The present invention is also directed to methods of providing personal hygiene products. In one exemplary embodiment of the present invention, the method of providing a personal hygiene product comprises providing a packaged solid personal hygiene product comprising (i) particulate personal hygiene product in the form of one or more tablets, one or more compressed granules, or a plurality of loose powder particles within (ii) a packaging material that encloses and protects the particulate personal hygiene product, the particulate personal hygiene product being convertible into a personal hygiene product when combined with a predetermined amount of liquid, such as water. The ready-to-use packaged solid personal hygiene products contain less than about 5.0 wt % water (or less than 4.0 wt % water, or less than 3.0 wt % water, or less than 2.0 wt % water, or less than 1.0 wt % water, or less than 0.5 wt % water) and therefore must be diluted with a liquid, such as water, in order to having a viscosity similar to that of conventional personal hygiene products in the form of pre-packaged creams, pastes or gels.

Methods of providing personal hygiene products, for example, to a user or consumer, may comprise offering for sale the packaged solid personal hygiene product comprising (i) particulate personal hygiene product in the form of one or more tablets, one or more compressed granules, or a plurality of loose powder particles within (ii) a packaging material. Methods of providing personal hygiene products may further comprise a number of additional method steps including, but are not limited to, forming the particulate personal hygiene product; any of the above-mentioned method steps of making the particulate personal hygiene product and/or the packaging material for the particulate personal hygiene product; packaging the particulate personal hygiene product in packaging material; combining a predetermined amount of liquid with the particulate personal hygiene product to form a personal hygiene product comprising a gel, paste or cream; advertising and/or marketing the commercial availability of the packaged solid personal hygiene product; shipping/mailing the packaged solid personal hygiene product to a shipping/mailing destination; and applying the gel, paste or cream onto at least one of teeth, hair and skin.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

### Preparation of a Shaving Cream Particulate Product

An exemplary particulate personal hygiene product in the form of a tablet was prepared as follows. The exemplary particulate personal hygiene product had a composition as shown in Table 1 below.

TABLE 1
Shaving Cream Particulate Personal Hygiene Product Components

| Composition | | | | |
|---|---|---|---|---|
| Components | Source | Amount | | Wt % |
| Soda Ash (LT) | Sigma-Aldrich Co. | 83 | g | 32 |
| | (St. Louis, MO) | | | |
| Citric Acid | Sigma-Aldrich Co. | 61.8 | g | 28 |
| | (St. Louis, MO) | | | |
| CRODALAN | Croda Inc. (Edison, NJ) | 3 | ml | 1 |
| AWS ™ | | | | |
| Glycerine | Sigma-Aldrich Co. | 10 | ml | 4 |
| | (St. Louis, MO) | | | |
| METHOCEL ™ | Dow Chemical | 25 | g | 10 |
| 40-202 | (Midland, MI) | | | |
| Stearic Acid | Sigma-Aldrich Co. | 65 | g | 25 |
| sodium salt | (St. Louis, MO) | | | |

The particulate personal hygiene product was formed by blending soda ash, citric acid, and stearic acid salt, and then adding CRODALAN AWS™ and glycerine liquids so that the CRODALAN AWS™ and glycerine liquids were absorbed onto the particles of soda ash, citric acid, and stearic acid salt. After blending, the particulate METHOCEL™ 40-202 was added to the mixture and further blended to form a particulate personal hygiene tablet. The resulting particulate personal hygiene product was formed into tablets having a tablet weight of about 5 g/tablet using conventional tablet-forming techniques.

A single tablet was combined with 20 ml of water to form enough shaving cream for a single shaving for a single user.

### EXAMPLE 2

### Preparation of a Shaving Cream Particulate Product

An exemplary particulate personal hygiene product in the form of a tablet was prepared as described in Example 1 above except PRIMOJEL® crosslinked potato starch (Campina Corp. (Netherlands) was used in place of the METHOCEL™ 40-202. The resulting tablets had a tablet weight of about 5 g/tablet.

A single tablet was combined with 25 ml of water to form enough shaving cream for a single face shaving.

### EXAMPLE 3

### Preparation of Shampoo/Body Wash Powder

An exemplary particulate personal hygiene product (sample 1) in the form of loose (i.e., free flowing) powder was prepared as follows. The exemplary particulate personal hygiene product had a composition as shown in Table 2 below.

TABLE 2
Shampoo/Body Wash Particulate Personal Hygiene Product
Components

| | | | |
|---|---|---|---|
| Composition | | Amount | |
| Components | Source | (mg) | Wt % |

| | | | |
|---|---|---|---|
| PRIMOJEL ® | Generichem | 4700 | 78.33 |
| crosslinked | (Totowa, NJ) | | |
| potato starch | | | |
| Green-Apple | Arylessence | 25.8 | 0.43 |
| | (Marietta, GA) | | |
| Sodium | Natrium | 100 | 1.67 |
| Bicarbonate | (Cortland, NY) | | |
| PEG 3350 | Union Carbide | 0 | 0 |
| | (Houston, TX) | | |
| NINOL ® C-5 | Stepan | 367 | 6.12 |
| | (Northfield, IL) | | |
| MALTRIN ® | Univar USA | 600 | 10.00 |
| M510 | (Bellevue, WA) | | |
| Jojoba Oil | Arista | 7 | 0.12 |
| | (Wilton, CT) | | |
| Octyl Palmitate | Stepan | 0 | 0 |
| | (Northfield, IL) | | |
| Sodium Lauryl | Stepan | 200 | 3.33 |
| Sulfate | (Northfield, IL) | | |
| FD&C | Prime Ingredients | 0.1 | 0.002 |
| Green #3 | (Saddle Brook, NJ) | | |
| FD&C | Prime Ingredients | 0.1 | 0.002 |
| Yellow #5 | (Saddle Brook, NJ) | | |

| | | | |
|---|---|---|---|
| TOTAL | 6000 | mg | 100.00% |

The particulate personal hygiene product was formed by mixing the liquid components (i.e., Green-Apple, NINOL® C-5, Jojoba oil and octyl palmitate (when present)) with sodium bicarbonate so as to be absorbed onto surfaces of the sodium bicarbonate particles. The MALTRIN® M510 was then blended into the mixture in order to form a mixture having a more free flowing consistency. Then the following solid components (when present) were added to the mixture: PEG 3350, sodium lauryl sulfate, FD&C Green #3, and FD&C Yellow #5.

After blending, particulate PRIMOJEL® crosslinked potato starch was added to the mixture and further blended to form a particulate personal hygiene product in form of loose powder.

6.0 g of the resulting particulate personal hygiene product (sample 1) was combined with 30.0 ml of water to form a general use shampoo/body wash gel.

### EXAMPLE 4

### Preparation of Shampoo/Body Wash Products

Two exemplary particulate personal hygiene products (samples 2-3) in the form of loose (i.e., free flowing) powder were prepared as described in Example 3 above. A third exemplary particulate personal hygiene product (sample 4) in the form of tablets was also formed. The exemplary particulate personal hygiene products had compositions as shown in Table 3 below.

TABLE 3
Shampoo/Body Wash Particulate Personal Hygiene Product
Components

| | Sample 2 | | | Sample 3 | | | Sample 4 | | |
|---|---|---|---|---|---|---|---|---|---|
| Composition | Amount | | | Amount | | | Amount | | |
| Components | (mg) | | Wt % | (mg) | | Wt % | (mg) | | Wt % |
| PRIMOJEL ® | 4567 | | 76.12 | 4800 | | 80.00 | 3300 | | 55.00 |
| crosslinked | | | | | | | | | |
| potato starch | | | | | | | | | |
| Green-Apple | 25.8 | | 0.43 | 25.8 | | 0.43 | 25.8 | | 0.43 |
| Sodium | 100 | | 1.67 | 100 | | 1.67 | 100 | | 1.67 |
| Bicarbonate | | | | | | | | | |
| PEG 3350 | 0 | | 0 | 0 | | 0 | 1554 | | 25.90 |
| NINOL ® C-5 | 300 | | 5.00 | 467 | | 7.78 | 300 | | 5.00 |
| MALTRIN ® | 600 | | 10.00 | 600 | | 10.00 | 600 | | 10.00 |
| M510 | | | | | | | | | |
| Jojoba Oil | 7 | | 0.12 | 7 | | 0.12 | 0 | | 0 |
| Octyl Palmitate | 200 | | 3.33 | 0 | | 0 | 0 | | 0 |
| Sodium Lauryl | 200 | | 3.33 | 0 | | 0 | 120 | | 2.00 |
| Sulfate | | | | | | | | | |
| FD&C | 0.1 | | 0.002 | 0.1 | | 0.002 | 0.1 | | 0.002 |
| Green #3 | | | | | | | | | |
| FD&C | 0.1 | | 0.002 | 0.1 | | 0.002 | 0.1 | | 0.002 |
| Yellow #5 | | | | | | | | | |
| TOTALS | 6000 | mg | 100% | 6000 | mg | 100% | 6000 | mg | 100% |

Sample 2 was formulated to produce a powder having an “enhanced skin feel.” As used herein, the term “enhanced skin feel” refers to how the product fells on a user's skin during use and after being rinsed away. The term “enhanced skin feel” is being used in a manner identical to the generally accepted meaning used in the skin product industry. 6.0 g of sample 2 was combined with 30.0 ml of water to form a shampoo/body wash gel having an “enhanced skin feel.”

Sample 3 was formulated to produce a powder having a tearless property. 6.0 g of sample 3 was combined with 30.0 ml of water to form a shampoo/body wash gel that did not cause tearing when used as a shampoo (i.e., did nor cause eye irritation when used as a shampoo).

Once sample 4 was formed into a mixture of particles using the method steps described in Example 3 above, the particles were formed into tablets using conventional tablet-forming techniques. Tablets were formed so as to have a tablet weight of about 6.0 g/tablet. When combined with 30.0 ml of water, a single tablet formed a general use shampoo/body wash gel.

### EXAMPLE 5

### Preparation of Toothpaste Powder

An exemplary particulate personal hygiene product (sample 5) in the form of loose (i.e., free flowing) powder was prepared as follows. The exemplary particulate personal hygiene product had a composition as shown in Table 4 below.

TABLE 4
Toothpaste Particulate Personal Hygiene Product Components

| Composition | | Amount | |
|---|---|---|---|
| Components | Source | (mg) | Wt % |
| PRIMOJEL ® | Generichem | 960 | 96.00 |
| crosslinked | (Totowa, NJ) | | |
| potato starch | | | |
| Peppermint or | Arylessence | 6 | 0.60 |
| Spearmint | (Marietta, GA) | | |
| Flavor | | | |
| Sodium | Natrium | 7.8 | 0.78 |
| Bicarbonate | (Cortland, NY) | | |
| PEG 3350 | Union Carbide | 0 | 0 |
| | (Houston, TX) | | |
| Xylitol | Univar USA | 0 | 0 |
| | (Bellevue, WA) | | |
| Sucralose | Univar USA | 6 | 0.60 |
| | (Bellevue, WA) | | |
| Sodium | Univar USA | 0 | 0 |
| Fluoride | (Bellevue, WA) | | |
| Sodium | Brenntag | 0 | 0 |
| Percarbonate | (Mulheim/Ruhr, Germany) | | |
| Sodium Lauryl | Stepan | 20 | 2.00 |
| Sulfate | (Northfield, IL) | | |
| FD&C | Prime Ingredients | 0.1 | 0.01 |
| Blue #1 | (Saddle Brook, NJ) | | |
| FD&C | Prime Ingredients | 0.1 | 0.01 |
| Yellow #5 | (Saddle Brook, NJ) | | |

| | | | |
|---|---|---|---|
| TOTAL | 1000 | mg | 100.00% |

The particulate personal hygiene product was formed by mixing the liquid component (i.e., peppermint or spearmint flavor) with sodium bicarbonate so as to be absorbed onto surfaces of the sodium bicarbonate particles. The following solid components (when present) were then added to the mixture: PEG 3350, xylitol, sucralose, sodium fluoride, sodium percarbonate, sodium lauryl sulfate, FD&C Blue #1, and FD&C Yellow #5 to form a solid mixture.

After blending, particulate PRIMOJEL® crosslinked potato starch was added to the mixture and further blended to form a particulate personal hygiene product in form of loose powder.

1.0 g of the resulting particulate personal hygiene product (sample 5) was combined with 5.0 ml of water to form a general use toothpaste.

### EXAMPLE 6

### Preparation of Toothpaste Products

Two exemplary particulate personal hygiene products (samples 6-7) in the form of loose (i.e., free flowing) powder were prepared as described in Example 5 above. A third exemplary particulate personal hygiene product (sample 8) in the form of tablets was also formed. The exemplary particulate personal hygiene products had compositions as shown in Table 5 below.

TABLE 5
Toothpaste Particulate Personal Hygiene Product Components

| | Sample 6 | | | Sample 7 | | | Sample 8 | | |
|---|---|---|---|---|---|---|---|---|---|
| Composition | Amount | | | Amount | | | Amount | | |
| Components | (mg) | | Wt % | (mg) | | Wt % | (mg) | | Wt % |
| PRIMOJEL ® | 949 | | 94.90 | 960 | | 96.00 | 750 | | 75.00 |
| crosslinked | | | | | | | | | |
| potato starch | | | | | | | | | |
| Peppermint or | 6 | | 0.60 | 6 | | 0.60 | 6 | | 0.60 |
| Spearmint | | | | | | | | | |
| Flavor | | | | | | | | | |
| Sodium | 0 | | 0 | 0 | | 0 | 7.8 | | 0.78 |
| Bicarbonate | | | | | | | | | |
| PEG 3350 | 0 | | 0 | 0 | | 0 | 210 | | 21.00 |
| Xylitol | 8.8 | | 0.88 | 0 | | 0 | 0 | | 0 |
| Sucralose | 6 | | 0.60 | 6 | | 0.60 | 6 | | 0.60 |
| Sodium | 0 | | 0 | 7.8 | | 0.78 | 0 | | 0 |
| Fluoride | | | | | | | | | |
| Sodium | 10 | | 1.00 | 0 | | 0 | 0 | | 0 |
| Percarbonate | | | | | | | | | |
| Sodium Lauryl | 20 | | 2.00 | 20 | | 2.00 | 20 | | 2.00 |
| Sulfate | | | | | | | | | |
| FD&C | 0.1 | | 0.01 | 0.1 | | 0.01 | 0.1 | | 0.01 |
| Blue #1 | | | | | | | | | |
| FD&C | 0.1 | | 0.01 | 0.1 | | 0.01 | 0.1 | | 0.01 |
| Yellow #5 | | | | | | | | | |
| TOTALS | 1000 | mg | 100% | 1000 | mg | 100% | 1000 | mg | 100% |

Sample 6 was formulated to produce a toothpaste powder. 1.0 g of sample 6 was combined with 5.0 ml of water to form a toothpaste having teeth whitening and cavity fighting properties.

Sample 7 was formulated to produce a toothpaste powder. 1.0 g of sample 6 was combined with 5.0 ml of water to form a toothpaste having fluoride.

Once sample 8 was formed into a mixture of particles using the method steps described in Example 5 above, the particles were formed into tablets using conventional tablet-forming techniques. Tablets were formed so as to have a tablet weight of about 1.0 g/tablet. When combined with 5.0 ml of water, a single tablet formed a general use toothpaste.

### EXAMPLE 7

### Preparation of Shaving Powder

An exemplary particulate personal hygiene product (sample 9) in the form of loose (i.e., free flowing) powder was prepared as follows. The exemplary particulate personal hygiene product had a composition as shown in Table 6 below.

TABLE 6
Shaving Particulate Personal Hygiene Product Components

| Composition | | Amount | |
|---|---|---|---|
| Components | Source | (mg) | Wt % |
| PRIMOJEL ® | Generichem | 4500 | 90.00 |
| crosslinked | (Totowa, NJ) | | |
| potato starch | | | |
| Green-Apple | Arylessence | 25 | 0.50 |
| | (Marietta, GA) | | |
| Sodium | Natrium | 7.8 | 0.16 |
| Bicarbonate | (Cortland, NY) | | |
| PEG 3350 | Union Carbide | 0 | 0 |
| | (Houston, TX) | | |
| 745GC skin | Stepan | 367 | 7.34 |
| feel enhancer | (Northfield, IL) | | |
| MALTRIN ® | Univar USA | 0 | 0 |
| M510 | (Bellevue, WA) | | |
| Axhenol DHSC | Pure Bioscience | 0 | 0 |
| | (El Cajon, CA) | | |
| Octyl Palmitate | Stepan | 0 | 0 |
| | (Northfield, IL) | | |
| Sodium Lauryl | Stepan | 100 | 2.00 |
| Sulfate | (Northfield, IL) | | |
| FD&C | Prime Ingredients | 0.1 | 0.002 |
| Blue #1 | (Saddle Brook, NJ) | | |
| FD&C | Prime Ingredients | 0.1 | 0.002 |
| Yellow #5 | (Saddle Brook, NJ) | | |

| | | | |
|---|---|---|---|
| TOTAL | 5000 | mg | 100.00% |

The particulate personal hygiene product was formed by mixing the liquid components (i.e., Green-Apple, and octyl palmitate (when present)) with sodium bicarbonate so as to be absorbed onto surfaces of the sodium bicarbonate particles. The MALTRIN® M510 (when present) was then blended into the mixture in order to form a mixture having a more free flowing consistency. Then, the following solid components (when present) were added to the mixture: PEG 3350, 745GC compound, Axhenol DHSC, sodium lauryl sulfate, FD&C Blue #1, and FD&C Yellow #5 to form a solid mixture.

After blending, particulate PRIMOJEL® crosslinked potato starch was added to the mixture and further blended to form a particulate personal hygiene product in form of loose powder.

5.0 g of the resulting particulate personal hygiene product (sample 9) was combined with 25.0 ml of water to form a general use shaving gel.

### EXAMPLE 8

### Preparation of Shaving Products

Two exemplary particulate personal hygiene products (samples 10-11) in the form of loose (i.e., free flowing) powder were prepared as described in Example 7 above. A third exemplary particulate personal hygiene product (sample 12) in the form of tablets was also formed. The exemplary particulate personal hygiene products had compositions as shown in Table 7 below.

TABLE 7
Shaving Particulate Personal Hygiene Product Components

| | Sample 10 | | | Sample 11 | | | Sample 12 | | |
|---|---|---|---|---|---|---|---|---|---|
| Composition | Amount | | | Amount | | | Amount | | |
| Components | (mg) | | Wt % | (mg) | | Wt % | (mg) | | Wt % |
| PRIMOJEL ® | 4174 | | 83.48 | 4307 | | 86.14 | 3000 | | 60.00 |
| crosslinked | | | | | | | | | |
| potato starch | | | | | | | | | |
| Green-Apple | 25.8 | | 0.52 | 25.8 | | 0.52 | 25 | | 0.50 |
| Sodium | 0 | | 0 | 0 | | 0 | 7.8 | | 0.16 |
| Bicarbonate | | | | | | | | | |
| PEG 3350 | 0 | | 0 | 0 | | 0 | 1400 | | 28.00 |
| 745GC skin | 500 | | 10.00 | 367 | | 7.34 | 367 | | 7.34 |
| feel enhancer | | | | | | | | | |
| MALTRIN ® | 0 | | 0 | 0 | | 0 | 100 | | 2.00 |
| M510 | | | | | | | | | |
| Axhenol | 0 | | 0 | 200 | | 4.00 | 0 | | 0 |
| DHSC | | | | | | | | | |
| Octyl Palmitate | 200 | | 4.00 | 0 | | 0 | 0 | | 0 |
| Sodium Lauryl | 100 | | 2.00 | 100 | | 2.00 | 100 | | 2.00 |
| Sulfate | | | | | | | | | |
| FD&C | 0.1 | | 0.002 | 0.1 | | 0.002 | 0.1 | | 0.002 |
| Blue #1 | | | | | | | | | |
| FD&C | 0.1 | | 0.002 | 0.1 | | 0.002 | 0.1 | | 0.002 |
| Yellow #5 | | | | | | | | | |
| TOTALS | 1000 | mg | 100% | 1000 | mg | 100% | 1000 | mg | 100% |

Sample 10 was formulated to produce a shaving powder having an “enhanced skin feel.” 5.0 g of sample 10 was combined with 25.0 ml of water to form a shaving gel having an “enhanced skin feel.”

Sample 11 was formulated to produce a powder having antimicrobial properties. 5.0 g of sample 11 was combined with 25.0 ml of water to form a shaving gel having antimicrobial properties.

Once sample 12 was formed into a mixture of particles using the method steps described in Example 7 above, the particles were formed into tablets using conventional tablet-forming techniques. Tablets were formed so as to have a tablet weight of about 5.0 g/tablet. When combined with 25.0 ml of water, a single tablet formed a general use shaving gel.

### EXAMPLE 9

### Preparation of Personal Hygiene Kits

Each of the exemplary particulate personal hygiene products formed in Examples 3-8 (samples 1-12) were packaged in separate individual bottles having a bottle volume and a removable cap. Each removable cap was designed to be used as a mixing container for forming a resulting personal hygiene product from the individual packaged particulate personal hygiene products. Various combinations of individually packaged particulate personal hygiene products comprising (i) at least one shampoo/body wash product from Examples 3-4, (ii) at least one toothpaste product from Examples 5-6, and (iii) at least one shaving product from Examples 7-8 were assembled together and further packaged in a kit packaging material to form various travel kits.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A method of providing a personal hygiene product, said method comprising:
providing a packaged solid personal hygiene product comprising (i) particulate personal hygiene product in the form of one or more tablets, one or more compressed granules, or a plurality of loose powder particles within (ii) a packaging material that encloses and protects the particulate personal hygiene product, the particulate personal hygiene product being convertible into a personal hygiene product when combined with a predetermined amount of liquid.

2. The method of claim 1, wherein said providing step comprises offering for sale the packaged solid personal hygiene product.

3. The method of claim 1, wherein said providing step comprises:
forming the particulate personal hygiene product; and
packaging the particulate personal hygiene product in the packaging material.

4. The method of claim 1, further comprising:
combining the predetermined amount of liquid with the particulate personal hygiene product to form a personal hygiene product comprising a gel, paste or cream.

5. The method of claim 4, further comprising:
applying the gel, paste or cream onto at least one of teeth, hair and skin.

6. The method of claim 1, wherein the liquid comprises water.

7. The method of claim 1, wherein the particulate personal hygiene product comprises:
at least one thickening agent in an amount greater than 10 wt % based on a total weight of the particulate personal hygiene product;
at least one active ingredient selected from the group consisting of a tooth cleaner, a tooth whitener, a breath freshener, an antimicrobial agent, a foaming agent, a surfactant, a hair conditioning agent, an anti-itch agent, a moisturizing agent, an emollient, an anti-wrinkle agent, an anti-aging agent, a sun-blocking agent, and combinations thereof;
optionally, at least one flavoring agent; and
optionally, at least one fragrance-producing agent.

8. The method of claim 7, wherein the at least one thickening agent is present in an amount ranging from about 15 wt % to about 99.9 wt % based on a total weight of the particulate personal hygiene product and is selected from the group consisting of naturally-occurring gums, starches, polyvinylpyrollidone, alkali metal starches, alkali metal starch glycolates, carboxycellulose compounds, cellulosic gums, xanthan gums, locust bean gums, and combinations thereof.

9. The method of claim 1, wherein the personal hygiene product comprises toothpaste, oral care products, shaving products, shampoo, hair conditioner, hair gel, skin lotion, or skin cream.

10. A particulate personal hygiene product in the form of one or more tablets, one or more compressed granules, or a plurality of loose powder particles, said particulate personal hygiene product comprising:
at least one thickening agent in an amount greater than 10 wt %;
at least one active ingredient in an amount ranging from greater than 0 to about 50 wt %, said at least one active ingredient being selected from the group consisting of a tooth cleaner, a tooth whitener, a breath freshener, an antimicrobial agent, a foaming agent, a surfactant, a hair conditioning agent, an anti-itch agent, a moisturizing agent, an emollient, an anti-wrinkle agent, an anti-aging agent, a sun-blocking agent, and combinations thereof;
optionally, at least one flavoring agent in an amount up to about 2.0 wt %; and
optionally, at least one fragrance-producing agent in an amount up to about 2.0 wt %;
wherein all weight percents are based on a total weight of said particulate personal hygiene product, and wherein said particulate personal hygiene product (i) forms a gel, paste or cream when blended with a predetermined amount of liquid, and (ii) comprises less than about 5.0 wt % water.

11. The particulate personal hygiene product of claim 10, wherein said at least one thickening agent is present in an amount ranging from about 15 wt % to about 99.9 wt % based on a total weight of the particulate personal hygiene product.

12. The particulate personal hygiene product of claim 10, wherein said at least one thickening agent is selected from the group consisting of naturally-occurring gums, starches, polyvinylpyrollidone, alkali metal starches, alkali metal starch glycolates, carboxycellulose compounds, cellulosic gums, xanthan gums, locust bean gums, crosslinked versions of any of these compounds or polymers, and combinations thereof.

13. The particulate personal hygiene product of claim 12, wherein said at least one thickening agent comprises a sodium starch glycolate formed from potato starch.

14. The particulate personal hygiene product of claim 10, wherein the gel, paste or cream comprises toothpaste, an oral care product, a shaving product, shampoo, hair conditioner, hair gel, skin lotion, or skin cream.

15. A packaged solid personal hygiene product comprising:
the particulate personal hygiene product of claim 14; and
packaging material that encloses and protects said particulate personal hygiene product.

16. A personal hygiene product in the form of a gel, paste or cream formed from the particulate personal hygiene product of claim 10.

17. A packaged solid personal hygiene product comprising:
particulate personal hygiene product capable of forming toothpaste, an oral care product, a shaving product, shampoo, hair conditioner, hair gel, skin lotion or skin cream when blended with a predetermined amount of liquid, said particulate personal hygiene product being in the form of one or more tablets, one or more compressed granules, or a plurality of loose powder particles; and
packaging material that encloses and protects said particulate personal hygiene product.

18. The packaged solid personal hygiene product of claim 17, wherein the particulate personal hygiene product comprises:
at least one thickening agent in an amount ranging from about 15 wt % to about 99.9 wt %;
at least one active ingredient in an amount ranging from greater than 0 to about 50 wt %, said at least one active ingredient being selected from the group consisting of a tooth cleaner, a tooth whitener, a breath freshener, an antimicrobial agent, a foaming agent, a surfactant, a hair conditioning agent, an anti-itch agent, a moisturizing agent, a sun-blocking agent, and combinations thereof;
optionally, at least one flavoring agent in an amount up to about 2.0 wt %;
optionally, at least one fragrance-producing agent in an amount up to about 2.0 wt %; and
less than about 5.0 wt % water;
wherein all weight percents are based on a total weight of said particulate personal hygiene product.

19. The packaged solid personal hygiene product of claim 17, wherein said packaging material provides the predetermined amount of liquid needed per package or per unit of particulate personal hygiene product so as to form one or more units of toothpaste, oral care product, shaving product, shampoo, hair conditioner, hair gel, skin lotion or skin cream.

20. The packaged solid personal hygiene product of claim 17, wherein the liquid comprises water.

21. The packaged solid personal hygiene product of claim 17, wherein said particulate personal hygiene product is present in an amount so as to provide from about 5 to about 50 units of personal hygiene product when combined with the predetermined amount of liquid.

22. A personal hygiene product in the form of a gel, paste or cream formed from the particulate personal hygiene product of the packaged solid personal hygiene product of claim 17.
